(19) United States
(12) Patent Application Publication
(10) Pub. No.: US 2013/0003016 A1
(43) Pub. Date: Jan. 3, 2013

(54) CALIBRATION DEVICE FOR USE WITH A FUNDUS CAMERA

(75) Inventors: Steven Feldon, Rochester, NY (US); William Fischer, Rochester, NY (US); Lana Jeanne Nagy, Higganum, CT (US)

(73) Assignee: UNIVERSITY OF ROCHESTER, Rochester, NY (US)

(21) Appl. No.: 13/521,217

(22) PCT Filed: Jan. 20, 2011
(86) PCT No.: PCT/US2011/021885

§ 371 Date: Jul. 9, 2012

Related U.S. Application Data
(60) Provisional application No. 61/296,882, filed on Jan. 20, 2010.

(51) Int. Cl. A61B 3/14

(52) U.S. Cl. 351/206; 351/246

(57) ABSTRACT

A calibration device for use with a fundus camera is described, including a mock retina having a curvature from a front portion to a back portion. The calibration device further includes a color model having a range of colors comparable to a range of colors of the fundus of an eye. The calibration device further can include a lens for magnification and is configured to facilitate generation of an image to evaluate and adjust an ophthalmic imaging system for features such as color, magnification, and resolution.

### CROSS-REFERENCE TO RELATED APPLICATIONS

This application is a national stage entry, under 35 U.S.C. §371, of International Application No. PCT/US2011/021885, filed on Jan. 20, 2011, and titled “Calibration Device for Use with a Fundus Camera,” which claims priority to U.S. Provisional Application Ser. No. 61/296,882, filed on Jan. 20, 2010, and titled, “Calibration Device for Use with a Fundus Camera,” both of which are hereby incorporated by reference herein.

### FIELD

The subject technology generally relates to ophthalmic imaging and, more particularly, to imaging using a fundus camera.

### BACKGROUND

A fundus camera, or retinal camera, is a specialized, low-power microscope with an attached camera, designed to photograph the fundus, or interior surface, of the eye, including the retina, optic disc, macula, and posterior pole. Fundus cameras are used by optometrists, ophthalmologists, and other trained medical professionals for diagnosis and monitoring progression of eye disease. Fundus photography may be combined with retinal angiography or used in screening programs.

### SUMMARY

Current fundus cameras suffer from the problem that aberrations of eye surfaces cause distortions and limit the resolution of the camera. In addition, colors of an object may be inconsistently detected or reproduced by the camera, and a degree of magnification by the camera lens system may be uncertain, due to calibration issues. Thus, there is a need for improved calibration of fundus cameras that avoids the shortcomings of prior calibration techniques.

In one aspect of the subject disclosure, a calibration device, for use with an eye fundus camera, comprises a body, an aperture, a mock retina, and a color model. The body may comprise a front, a back, and an inner portion extending at least partially between the front and the back. The aperture may be disposed at a front of the body and may permit light to enter the inner portion from outside the body. The mock retina may be disposed within the body and may be viewable from outside the body through aperture. A color model may be disposed in or on the mock retina, the color model may comprise a plurality of regions, each of the regions may have a color distinct from the color of another of the regions, wherein the color of each of the regions is substantially representative of a color of the fundus of a living eye.

In some embodiments, the inner portion comprises a cavity, and the mock retina is at the back of the cavity.

In some embodiments, the mock retina comprises a curvature that is concave from a front portion to a back portion of the mock retina.

In some embodiments, the body is substantially closed, permitting substantially no visible light to enter the inner cavity except through the aperture.

In some embodiments, the color of at least one of the regions is substantially representative of a color of the retina of a living eye.

In some embodiments, the color of at least one of the regions is substantially representative of a color of the optic nerve of a living eye.

In some embodiments, a lens positioned between the aperture and the mock retina, the lens magnifying or minifying the mock retina when the mock retina is viewed from outside the body.

In some embodiments, a mock iris may be disposed on the body and the mock iris may be configured to vary a size of the aperture may.

In some embodiments, the color of at least one of the regions has a red (R) value of between about 150 and 255 in an RGB color model.

In some embodiments, the color of at least one of the regions has a red (R) value of between about 180 and 255 in an RGB color model.

In some embodiments, the color of at least one of the regions has a red (R) value of between about 200 and 255 in an RGB color model.

In some embodiments, the color of at least one of the regions has a red (R) value of greater than about 160, and green (G) and blue (B) values of less than about 100, in an RGB color model.

In some embodiments, the color of at least one of the regions has a red (R) value of greater than about 200, and green (G) and blue (B) values of less than about 100, in an RGB color model.

In one aspect of the subject disclosure, a method of calibrating an eye fundus camera is disclosed. The method comprises positioning a body in front of a fundus camera, the body having an inner portion extending at least partially between a front and a back of the body. The method further comprises imaging with the camera, through an aperture at the front, an image model in the inner portion. The image model comprises a plurality of color regions, each of the regions having a color distinct from the color of another of the regions, wherein the color of each of the regions is substantially representative of a color of the fundus of a living eye. The method further comprises calibrating a color setting of the camera based on information derived from the imaging.

In some embodiments, the color of at least one of the regions is substantially representative of a color of the retina of a living eye.

In some embodiments, the color of at least one of the regions is substantially representative of a color of the optic nerve of a living eye.

In some embodiments, the image model comprises at least one of a dimensional or magnification scale, and further comprising calibrating a magnification setting of the camera based on information derived from the imaging.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology.

Accordingly, the drawings and detailed description are to be regarded as illustrative in nature and not as restrictive.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1 illustrates examples of the same human eye photographed with six different cameras.

FIG. 2 illustrates an example of a color space or gamut for the human eye.

FIG. 3 illustrates an example of a color chart.

FIG. 4A and 4B are diagrams illustrating examples of a perspective view of a calibration device for use with a fundus camera according to one aspect of the subject technology.

FIG. 5A and 5B are diagrams illustrating examples of an exploded view of the calibration device for use with a fundus camera according to one aspect of the subject technology.

FIG. 6 illustrates an example of an exploded view of a calibration device including a lens device according to one aspect of the subject technology.

FIG. 7A and 7B are examples of a top of the of the calibration device for use with a fundus camera according to one aspect of the subject technology.

FIG. 8 illustrates an example of a test target with RGB values of each square.

FIG. 9A is a diagram illustrating an example of a side view of a calibration device.

FIG. 9B is a diagram illustrating an example of a front view of the calibration device.

FIG. 10A is a diagram illustrating an example of a calibration device attached to a side bar to position the calibration device in front of a fundus camera.

FIG. 10B is a diagram illustrating an example of a calibration device attached to a chin rest to position the calibration device in front of the fundus camera.

FIG. 11A is a diagram illustrating an example of an unleveled calibration device.

FIG. 11B is a diagram illustrating an example of a leveled calibration device.

FIG. 12A is a diagram illustrating an example of an unleveled fundus camera.

FIG. 12B is a diagram illustrating an example of a leveled fundus camera.

FIG. 13 is a diagram illustrating an example of the fundus camera aligned and adjusted for working distance.

FIG. 14 is an example of a color calibration target according to one aspect of the subject technology.

FIG. 15 illustrates an exemplary process or method of calibrating an eye fundus camera.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like or similar components may be labeled with identical element numbers for ease of understanding or it may indicated in the disclosure that one component may be an example of a different component.

The optical system of the human eye has prompted the basic design specification for the camera. Light comes into the eye through the cornea, pupil, and lens at the front of the eye, as the lens of the camera lets light in. This light is then focused on the inside wall, or retina, of the eye, as on the film in a camera. Detectors that are distributed over the surface of the retina send impulses to the brain by the optic nerve, which connects the eye to the brain may detect this image, just as photographic film captures an image focused thereon.

The fundus camera includes an optical subsystem that illuminates an ocular fundus and collects the light reflected therefrom to produce an image of the ocular fundus. In particular, a fundus camera or retinal camera is a specialized low-power microscope with an attached camera designed to photograph the interior surface of the eye, including the retina, optic disc, macula, and posterior pole. However, fundus cameras are known to produce inconsistent color due to lack of adequate color management.

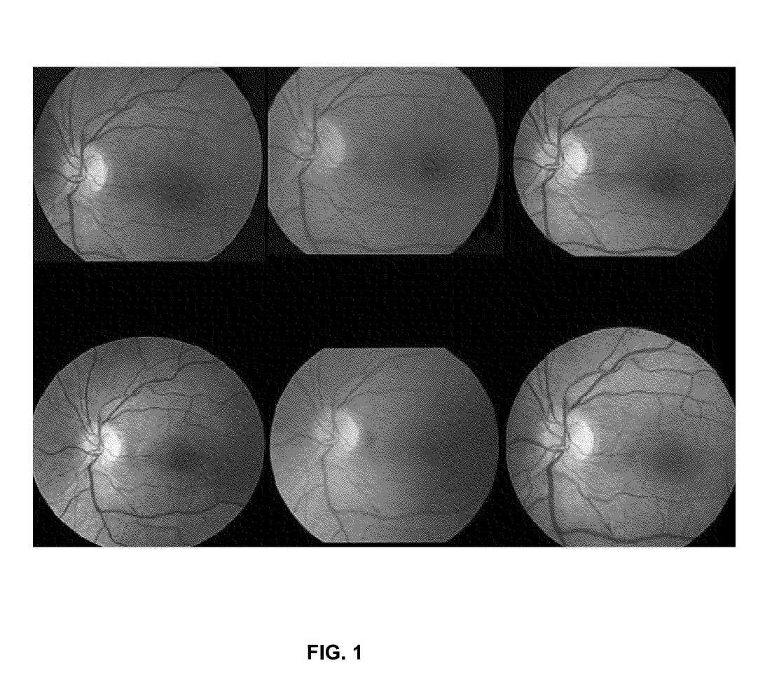

In the same way that different films and photo papers have different color characteristics, different camera sensors, computer monitors, and printers have different color characteristics. At Rochester Institute of Technology, one eye was photographed on six different fundus cameras and resulting in six different-looking images (FIG. 1). Diagnostically, it can be detrimental to have the same image look different at a capture station or image platform than it does on a review station. Many examination room computers and computer monitors are not designed for image viewing, and color shifts on the examination room monitor may mask the pathology captured on a station monitor. A scientific and diagnostic approach can be incorporated to address color management issues of cameras and it application to ophthalmic photography.

An understanding of color is important for addressing color management issues of cameras. There are three main factors that go into the color equation: a light source, an object and the observer. Changes in any of these three factors can change the outcome of a color equation. Different light sources can have different colors. For example, tungsten light appears yellow, fluorescent lights appear green, and many strobe lights appears bluish. The object in the color equation, and how it reacts to light, plays a role as well. A given object can interact with light to produce different colors depending on, for example, the angle at which the light contacts the object. The last factor in our color equation is the observer. Different observers may have different perceptions of color for various reasons.

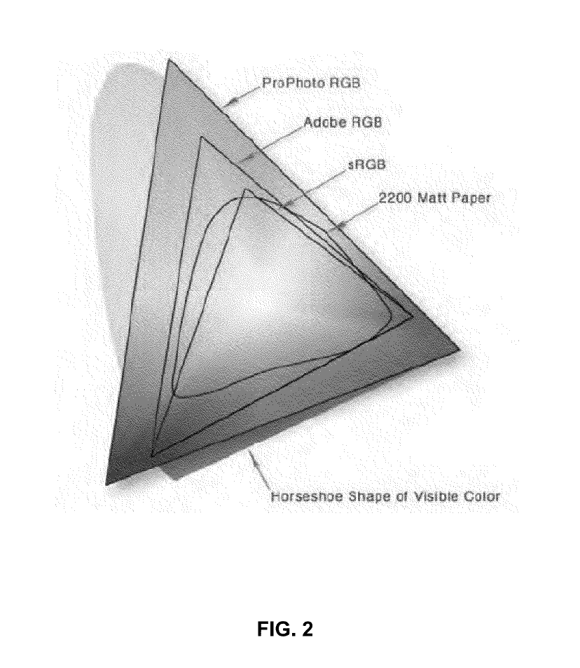

Part of understanding color also involves defining and describing color. In some applications, for example analog graphic design, Pantone color chips are used as a standard. In some applications, for example digital applications, a similar process may be applied that uses predetermined color values. Computers may quantify every color they display into very specific numerical values. These specific values may be used to manipulate colors and create a color-managed workflow. A digital device may be capable of reproducing specific colors or group of colors referred to as the device's color space or gamut. Referring to FIG. 2, the range of colors visible to the human eye is the largest color space. The visible colors may be reduced from one device, for example cameras, to the other. This is so because cameras, for example, may not be capable of capturing all the colors that can be captured or seen by an eye. Furthermore, monitors and software programs may not be able to display all the colors that a camera captured. Further yet, printers may have a very small set of colors they are capable of reproducing.

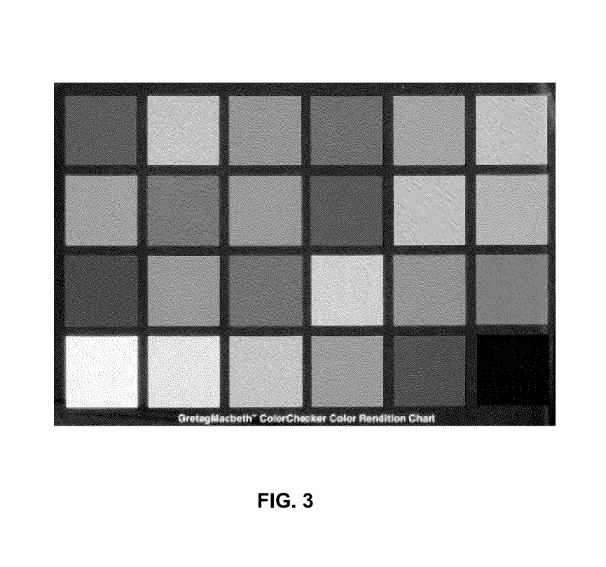

Focusing on the camera, every camera sensor manufacturer may have a different idea of what a correct set of colors may comprise. The manufacturer may optimize the color space a sensor is capable of reproducing based on their perception of the correct set of colors. Accordingly, the same eye photographed on different sensors can result in very different images. One solution for addressing this issue in commercial photography involves creating an International Color Consortium (ICC) profile using a color checker that has known numerical values, such as the color checker of FIG. 3. A photo may be taken of the color checker under the same conditions that may be used for future photos. However, the color checker photo may be required to have the same light source, the same flash power, the same f-stop, and the same ISO as the future photos taken. A profiling software may then be used to measure the numerical values the camera captured for the color checker and compare it to the known numerical values of the color checker. Given this information, the profiling software can create an input profile. However, this method is less than ideal for fundus photography, for a variety of reasons. Using the optics of the eye during fundus photography means that any media changes will affect the image. Thus, to overcome this issue it may be necessary to build a profile for each eye photographed. We would even need to re-create a profile for repeat patients since media opacities can progress. Perhaps the largest obstacle we encounter when trying to apply this solution is that we cannot put a color checker inside the eye. Thus, there is a need for improved calibration of fundus cameras that avoid the shortcomings and drawback of prior calibration techniques of fundus cameras.

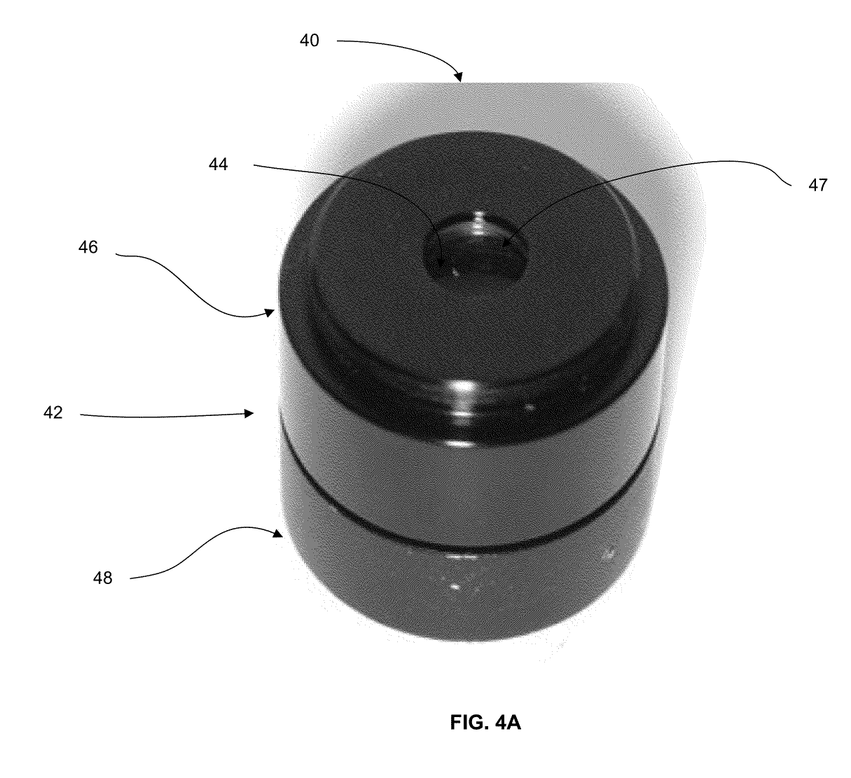

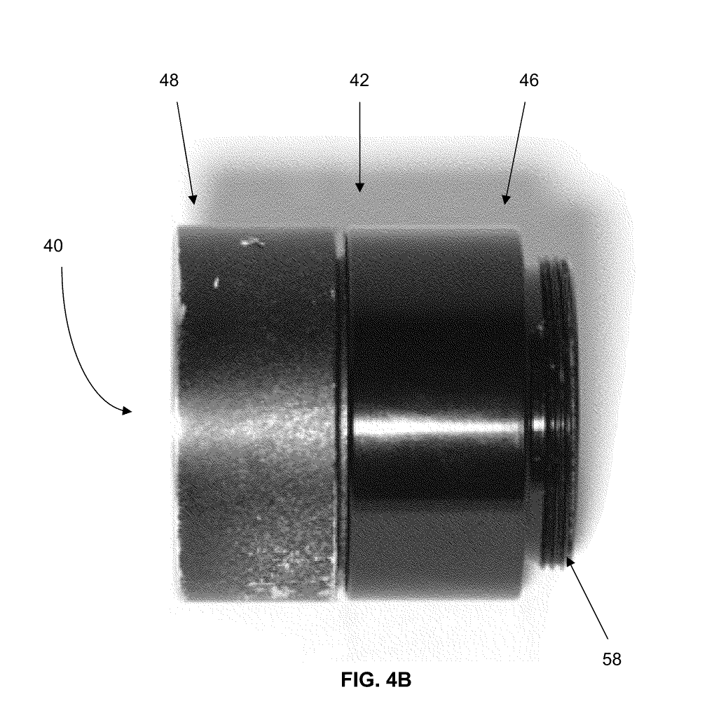

FIG. 4A and 4B are diagrams illustrating examples of a perspective view of a calibration device 40 for use with a fundus camera according to one aspect of the subject technology. In general, the calibration device may comprise body 42 and an aperture 44. In some embodiments, the body may comprise a single integrated body. In other embodiments, the body may comprise multiple sections configured to be coupled together. In some embodiments, the body 42 may comprise a front section 46, a back section 48 and an inner portion 47 extending at least partially between the front section 46 and the back section 48. The calibration device 40 may be configured to address inconsistent color produced in ophthalmic imaging systems such as fundus cameras. The calibration device may be referred to as a “test eye.” The test eye may emulate a human eye.

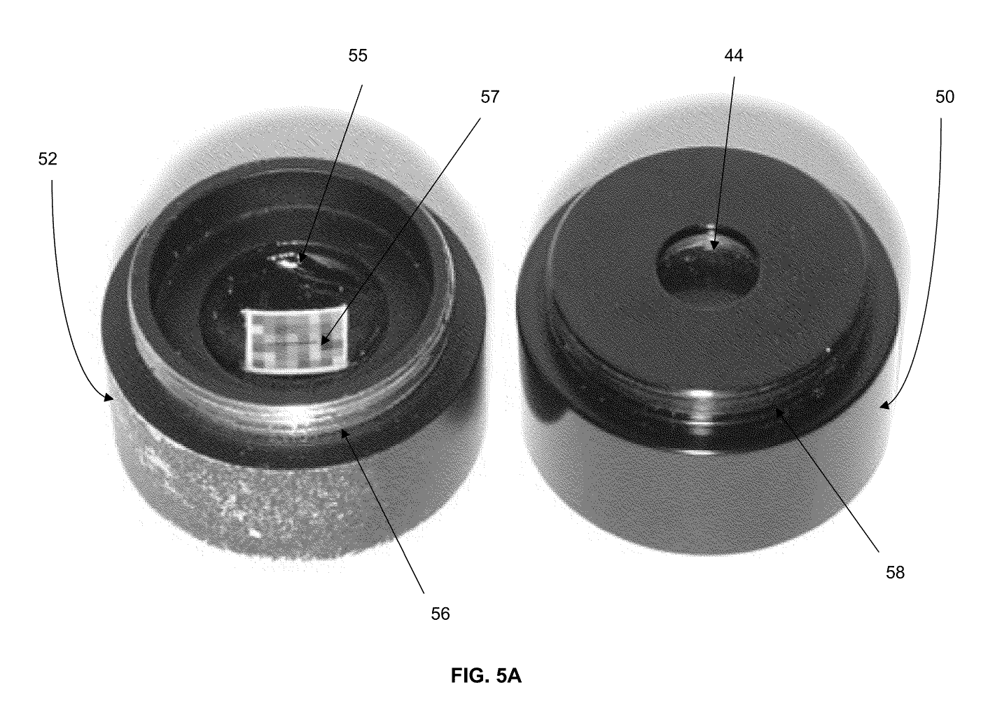

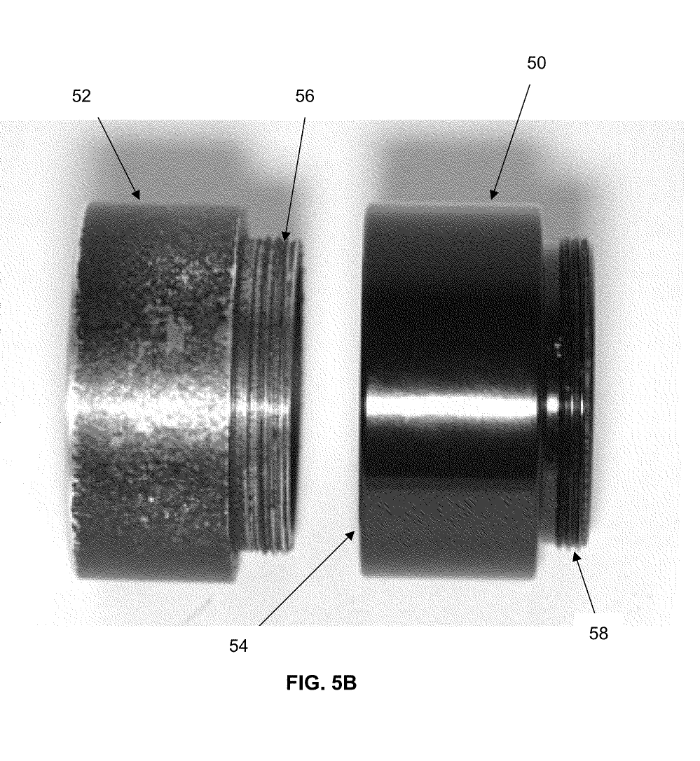

Referring to FIG. 5A and 5B, the body of the calibration device may comprise a first section 50 and a second section 52. The first section 50 may comprise a first connection portion 54 and the second section 52 may comprise a second connection portion 56. The first section 50 may be configured to be coupled to the second section 52 via the first and second connection portions 54 and 56, for example, as illustrated in FIG. 4A and 4B. In some embodiments, the first and second connection portions 54 and 56 may be threaded. In some embodiments, at least one of the first and second sections 50 and 52 may be configured to engage at least one section of an ophthalmic imaging system. For example, the first section 50 may comprise an engagement portion 58 configured to engage at least a portion of an ophthalmic imaging system. In some embodiments, the engagement portion 58 may be threaded. The aperture 44 may be disposed on the front section of the body and may permit light to enter the inner portion 47 from outside. In some embodiments, multiple apertures may be disposed on the body 42 and may permit light to enter the inner portion 47 from outside of the body 42. The mock retina 55 may be disposed in the body 42 of the calibration device 40. The mock retina 55 may be viewable from the outside of the body 42 through the aperture 44. The inner portion 47 may extend at least partially between the first section 52 and the second section 52. In some embodiments, the mock retina is disposed on the inner portion 47 associated with the second section 52. In some embodiments, the inner portion 47 comprises a cavity, and the mock retina 55 is at the back of the cavity. In some embodiments, the mock retina 55 comprises a curvature that is concave from a front portion to a back portion of the mock retina. In some embodiments, the body 42 is substantially closed, permitting substantially no visible light to enter the inner cavity except through the aperture 44.

Referring to FIG. 5A, the mock retina 55 of the calibration device 40 may comprise a color calibration target 57 and a magnification scale. The color calibration target 57 may be a part of a color portion or independent of the color portion. The magnification scale may be part of a lens or independent of the lens. In some embodiments, the magnification scale refers to a bar scale printed within the color test target or color calibration target 57. In some embodiments, the magnification scale can be calculated using a known size of a color calibration target 57 parameter such as a color calibration target 57 square, for example. The bar scale within the image may provide accurate measurement. The color calibration target 57 or color or test target may be used to correct for inaccurate color representation present in ophthalmic digital fundus images (“images” or “fundus images”). The magnification scale can be used to determine the exact size or magnification of an image. The test target may comprise colors having color frequency values. The colors and/or the color frequency values of the test target are developed by sampling tones present in multiple human eyes and reproducing the tones in the test target. In some embodiments, the color calibration target comprises but not limited to at least one of a Red Green Blue (RGB) color mode, a frequency representation of the color model, a wavelength representation of the color model, Cyan, Magenta, Yellow, and Key (black) (CMYK) color model, LAB color model, or a combination thereof. In some embodiments, the color of at least one of a region in the color calibration models is substantially representative of a color of the retina of a living eye. In some embodiments, the color of at least one of a region in the color calibration models is substantially representative of a color of the optic nerve of the living eye. In some embodiments, the color of at least one of a region in the color calibration models is substantially representative of a color of the living eye including at least one of the colors of the optic disc, vessels, retinal veins, macula, photoreceptors (including rods and cones), fovea, optic cup, retinal vessels, vasculature, optic nerve, retina and a combination thereof.

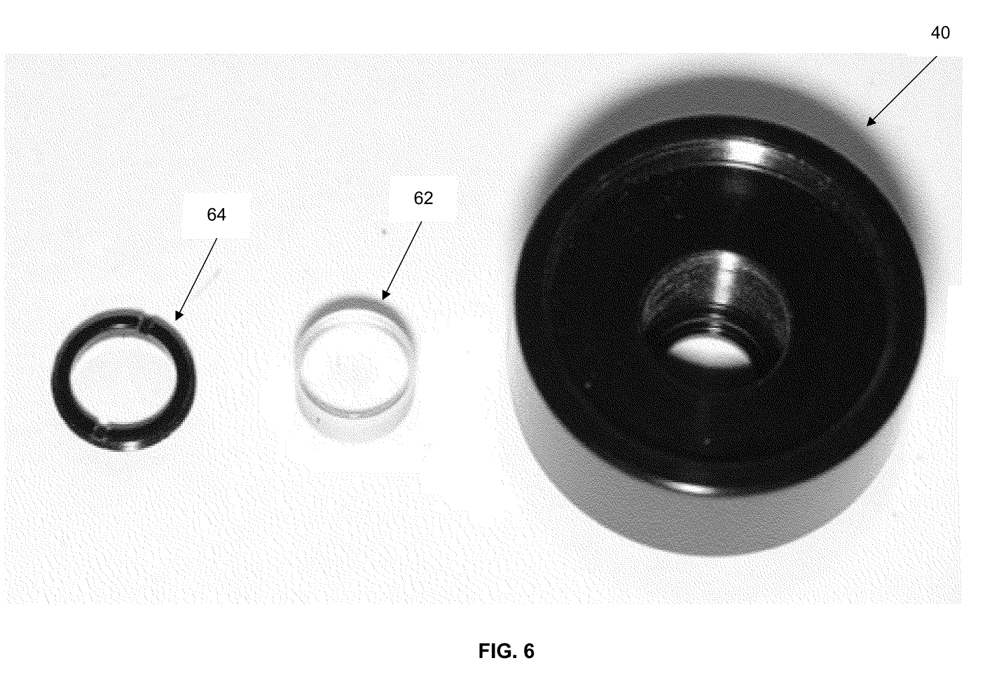

Referring to FIG. 6, the calibration device further illustrates an example of an exploded view of a calibration device 40. The calibration device 40 may comprise a lens 62 and a lens positioner 64. In some embodiments, the lens positioner 64 may be integrated into the body 42 of the calibration device 40. In some embodiments, the lens 62 may be integrated into the body 42 of the calibration device 40. In some embodiments, the lens positioner 64 may be configured to position the lens 62 within the body 42 of the calibration device 40. In some embodiments, the lens may be positioned between the aperture 44 and the mock retina 55. The lens 62 may be configured to magnify or minify the mock retina 55 when the mock retina 55 is viewed from outside the body. In some embodiments, the calibration device may comprise a mock iris (not shown) configured to vary the size of the aperture 44.

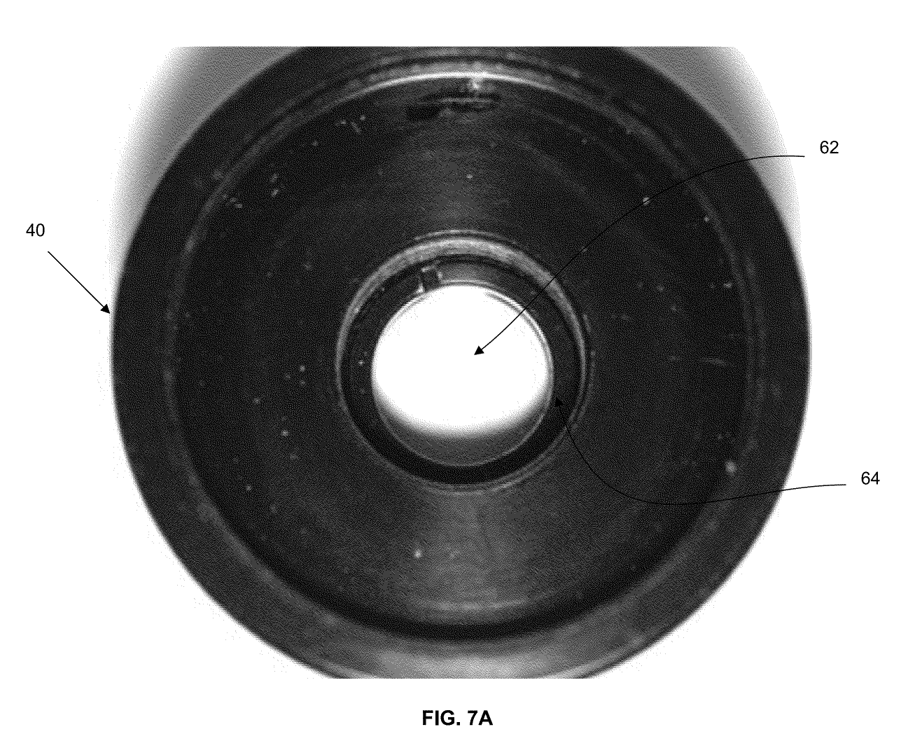

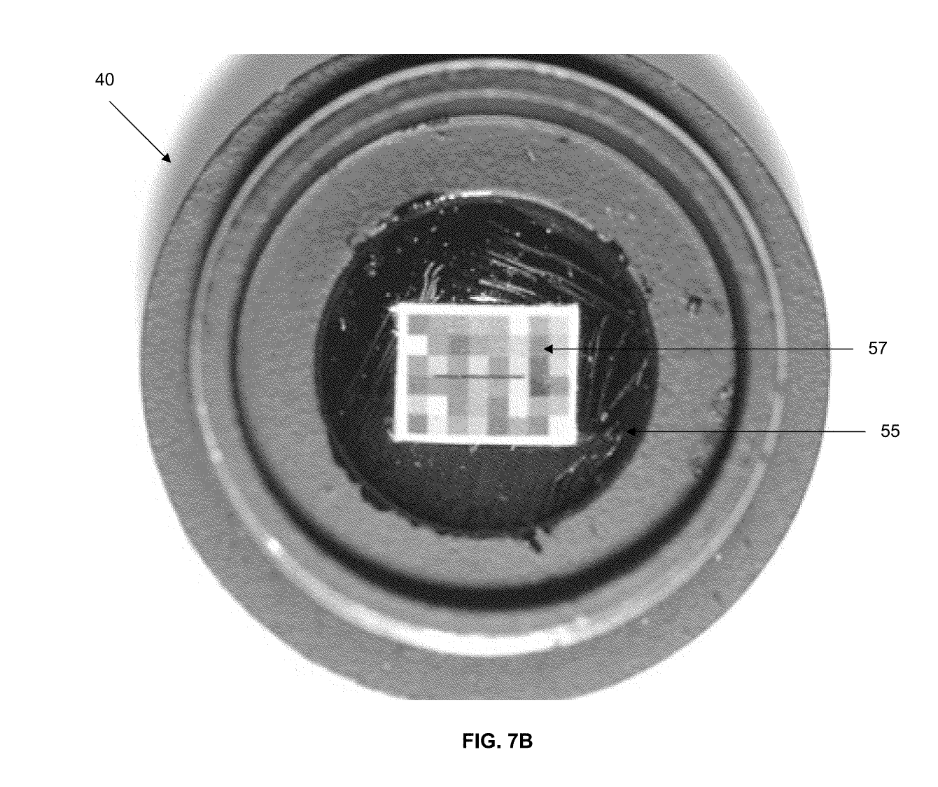

FIG. 7A and 7B are examples of a top view of the calibration device for use with a fundus camera according to one aspect of the subject technology. Referring to FIG. 7A, the calibration device further illustrates the lens 62 and the lens positioner 64 in position within the body 42 of the calibration device 40. Referring to FIG. 7B, the calibration device further illustrates the mock retina 55 and the color calibration target 57 positioned in the body 42 of the calibration device 40.

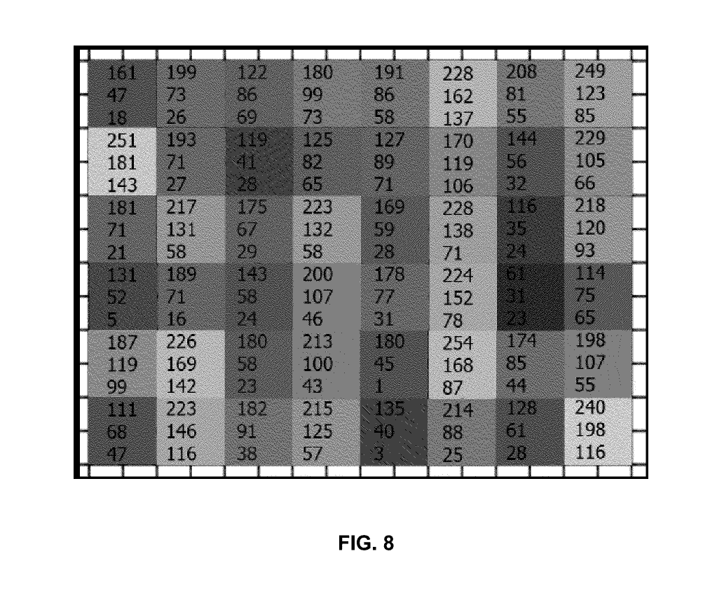

FIG. 8 illustrates an example of the RGB color calibration model with RGB values of each square or region. These RGB values may be values of a digital file. The color calibration target 57 may represent an optimum estimate, for example, of the RGB values to correct for retinal images across a diverse population. In some embodiments, several different RGB values may be implemented. In some embodiments, the color of at least one of the regions has a red (R) value of between about 150 and 255 in an RGB color model. In some embodiments, the color of at least one of the regions has a red (R) value of between about 180 and 255 in an RGB color model. In some embodiments, the color of at least one of the regions has a red (R) value of between about 200 and 255 in an RGB color model. In some embodiments, the color of at least one of the regions has a red (R) value of greater than about 160, and green (G) and blue (B) values of less than about 100, in an RGB color model. In some embodiments, the color of at least one of the regions has a red (R) value of greater than about 200, and green (G) and blue (B) values of less than about 100, in an RGB color model.

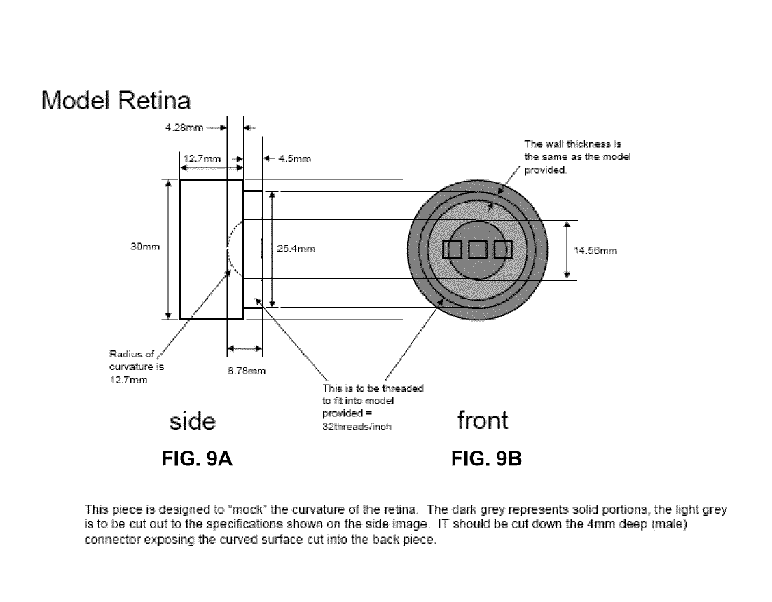

FIG. 9A is a diagram illustrating an example of a side view of a calibration device and FIG. 9B is a diagram illustrating an example of a front view of the calibration device. The calibration device comprises a mock retina, a color portion and a lens for magnification. The mock retina may comprise a curvature from a front side to a back side. The mock retina may be configured to emulate the retina of an eye. The color portion may comprise a range of colors comparable to the range of colors of the retinal tissues of the eye. The lens may be configured to emulate the cornea and lens of the eye. The calibration device may be configured to facilitate generation of an image to evaluate a fundus camera system for one of color, magnification and fundus camera system resolution. In some embodiments, the front side of the mock retina is configured to be positioned closer to the fundus camera than is the back side. The mock retina may be concave from the front side to the back side. In some embodiments, the calibration device comprises a mock iris, which may be configured to emulate the iris of a human eye in terms of having a variable aperture. In some embodiments, the calibration device comprises a mock pupil. The mock pupil may be configured to emulate the pupil of a human eye. In some embodiments, the calibration device comprises a mock fundus, which may be configured to emulate a fundus of the human eye in terms of curvature, color, and the presence of a mock vasculature. In some embodiments, the calibration device may be configured to be mounted to the fundus camera system in a manner that allows the fundus camera system to be adjusted such that the calibration device is level and parallel to the fundus camera system lens.

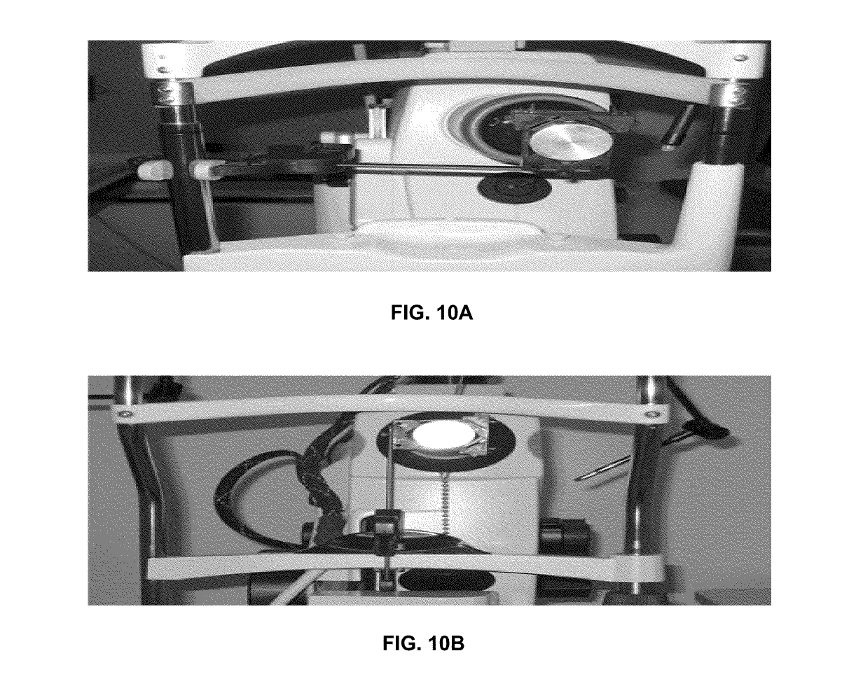

In some embodiments, the test eye (calibration device) is mounted or positioned in front of the ophthalmic imaging system in a manner that allows the test eye to be adjusted so that the test eye is level and parallel to, for example, the lens of the ophthalmic imaging system. FIG. 10A to 12B illustrate various positions of the calibration device mounted in front of the ophthalmic imaging device. Referring to FIG. 10A, the calibration device can be attached to a side bar to position the calibration device in front of a fundus camera. Referring to FIG. 10B, the calibration device can be attached to a chin rest, to position the calibration device in front of the fundus camera.

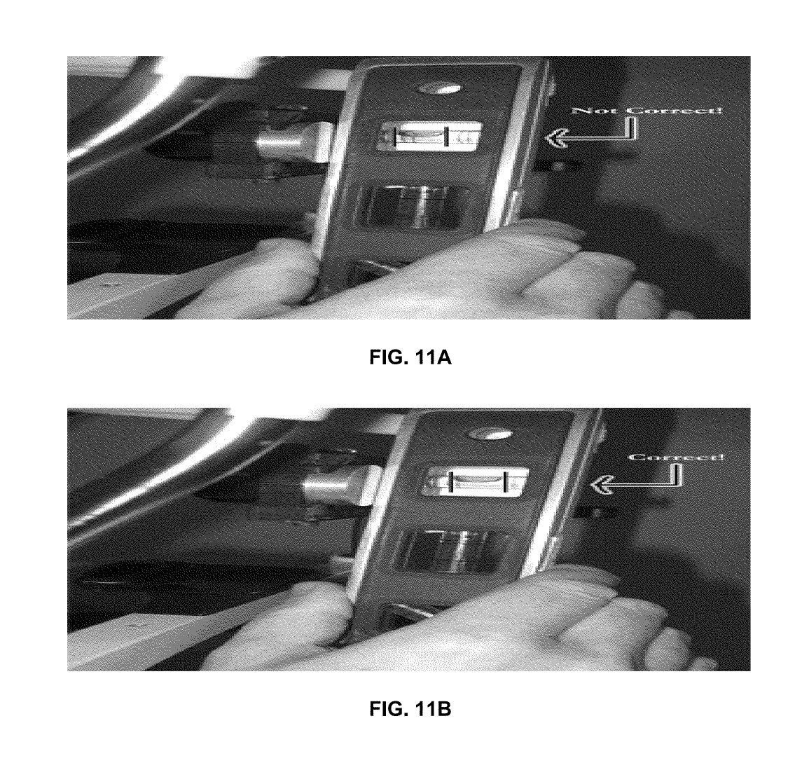

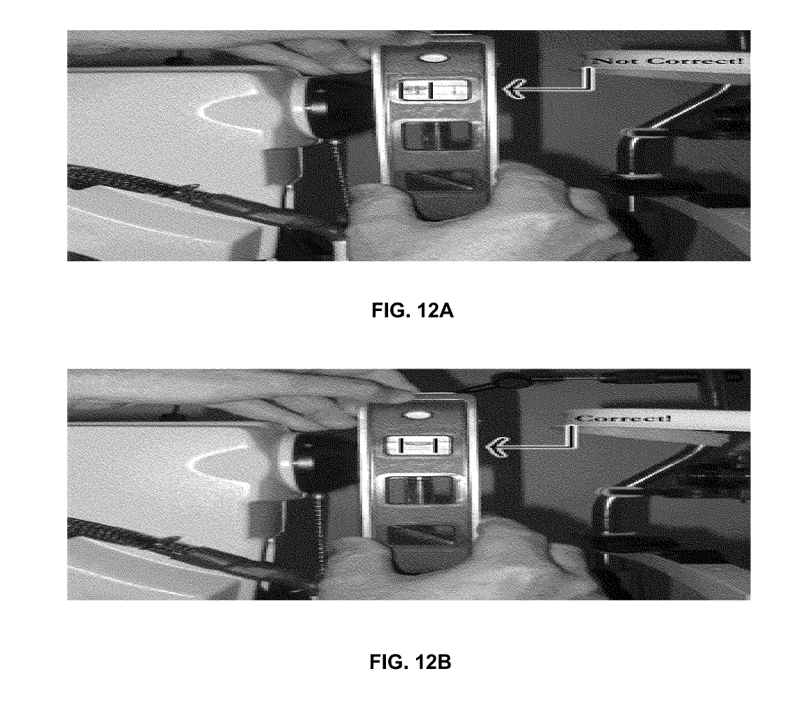

Referring to FIG. 11A, a diagram showing an unleveled calibration device positioned in front of an ophthalmic imaging system is illustrated. A leveling device may be used to adjust the test eye so that the back plane, for example, of the test eye is level vertically. Referring to FIG. 11B, a diagram showing a leveled calibration device positioned in front of an ophthalmic imaging system is illustrated. Referring to FIG. 12A, a diagram showing an unleveled fundus camera to be positioned in front of the test eye is illustrated. A leveling device may be used to adjust the fundus camera so that the fundus camera is leveled vertically. A leveling device may be held across the fundus camera lens mount, for example, and the camera tilt adjusted until the camera is level as illustrated in FIG. 12B. Leveling the camera and the test eye ensures that the objects, the camera and the calibration device are parallel to each other.

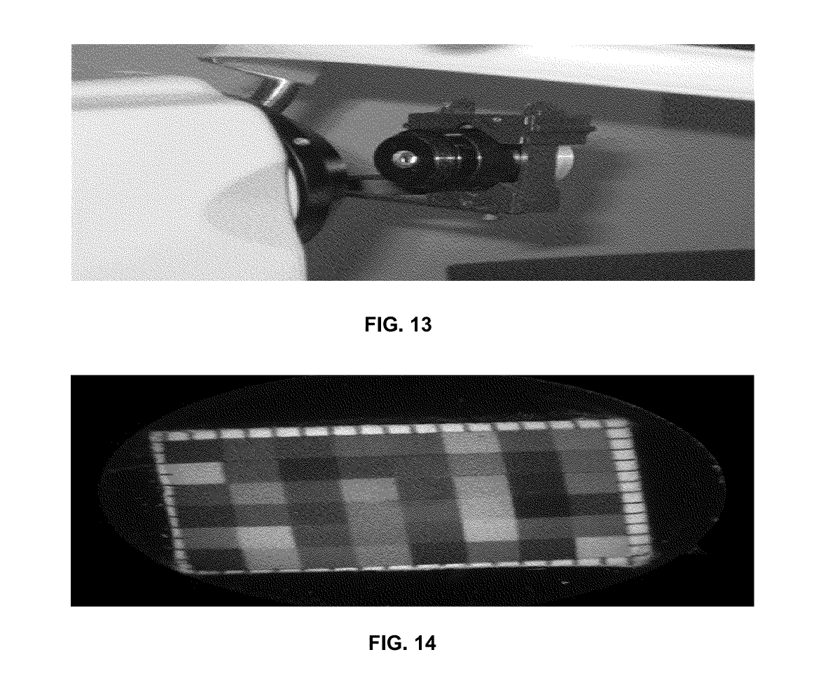

In some embodiments, the camera can be aligned in front of the eye and the camera swing-adjusted so that the camera and the calibration device are squared up to each other, as illustrated in FIG. 13. The working distance between the calibration device and the camera lens may be adjusted to produce a donut-like illumination on the lens of the calibration device. The camera may be focused and the working distance fine-tuned to improve the saturation and evenness of illumination of the image. Focusing the calibration device may include lining the fundus camera up with a pupil of the calibration device and focusing light having a donut-like feature on the pupil. This implementation may be similar to the steps of photographing a human eye. Proper alignment and positioning of the camera will enhance the illumination of the target. Better results may be acquired by avoiding a sharp focus of light in one area of the calibration device, by avoiding improper alignment and by avoiding tilting one end of the camera or the calibration device too close or too far. When the camera is lined up correctly, the camera can be focused on the target or image inside or on the calibration device and a picture of the image or target is taken. In some aspects, a determination of the exposure needed to produce an exposed image is determined in order to improve the results. In addition, multiple images of the target may be acquired for selection of the better images. These images acquired may be saved in a storage device. In some embodiments, when the exposure of the normal image is determined, multiple pictures may be taken. For example, a picture may be taken with the flash setting slightly increased and a picture may be taken with the flash setting slightly decreased to create a slightly exposed image and a slightly under exposed images. Each image can have detail in the highlights and shadows. One or more images of the target may be exported for further review. The one or more images may be exported to a computer, for example, or exported to a reading center, for example, for review. The one or more images may be saved as a .tiff file.

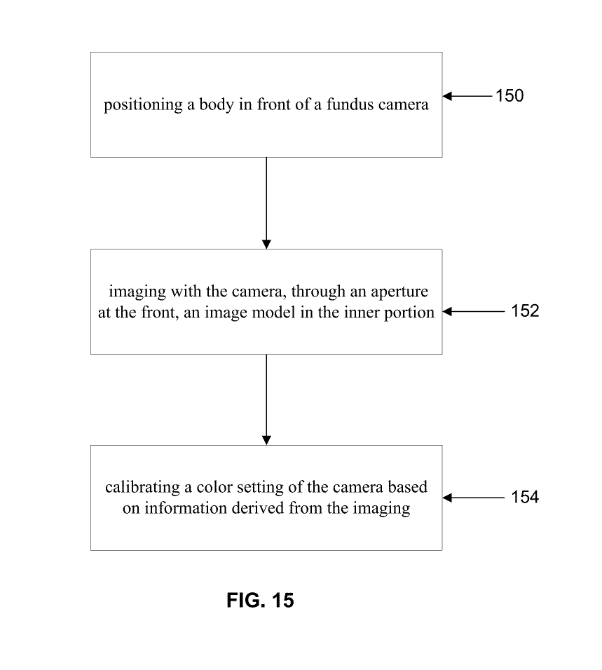

FIG. 15 illustrates an exemplary process or method of calibrating an eye fundus camera. In block 150, the method begins with positioning a body in front of a fundus camera, the body having an inner portion extending at least partially between a front and a back of the body. In block 152, the method includes imaging with the camera, through an aperture at the front, an image model in the inner portion. The image model comprises a plurality of color regions, each of the regions having a color distinct from the color of another of the regions, wherein the color of each of the regions is substantially representative of a color of the fundus of a living eye. The method then continues to block 154 where a color setting of the camera based on information derived from the imaging is calibrated.

The foregoing description is provided to enable a person skilled in the art to practice the various embodiments described herein. While the present subject technology has been particularly described with reference to the various figures and embodiments, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these embodiments will be readily apparent to those skilled in the art, and generic principles defined herein may be applied to other embodiments. Thus, many changes and modifications may be made to the subject technology, by one having ordinary skill in the art, without departing from the scope of the subject technology.

A phrase such as an “aspect” does not imply that such aspect is essential to the subject technology or that such aspect applies to all configurations or embodiments of the subject technology. A disclosure relating to an aspect may apply to all configurations, or one or more configurations. An aspect may provide one or more examples. A phrase such as an aspect may refer to one or more aspects and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as configuration may refer to one or more configurations and vice versa.

The word “exemplary” is used herein to mean “serving as an example or illustration.” Any aspect or design described herein as “exemplary” is not necessarily to be construed as preferred or advantageous over other aspects or designs.

A reference to an element in the singular is not intended to mean “one and only one” unless specifically stated, but rather “one or more.” The term “some” refers to one or more. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

What is claimed is:

1. A calibration device, for use with a eye fundus camera, comprising:
a body having a front, a back, and an inner portion extending at least partially between the front and the back;
an aperture at the front, permitting light to enter the inner portion from outside the body;
a mock retina in the body, viewable from outside the body through the aperture; and
a color model in or on the mock retina, the model comprising a plurality of regions, each of the regions having a color distinct from the color of another of the regions, wherein the color of each of the regions is substantially representative of a color of a fundus of a living eye.

2. The calibration device of claim 1, wherein the inner portion comprises a cavity, and the mock retina is at the back of the cavity.

3. The calibration device of claim 2, wherein the mock retina comprises a curvature that is concave from a front portion to a back portion of the mock retina.

4. The calibration device of claim 1, wherein the mock retina comprises a curvature that is concave from a front portion to a back portion of the mock retina.

5. The calibration device of claim 1, wherein the body is substantially closed, permitting substantially no visible light to enter the inner cavity except through the aperture.

6. The calibration device of claim 1, wherein the color of at least one of the regions is substantially representative of a color of the retina of a living eye.

7. The calibration device of claim 1, wherein the color of at least one of the regions is substantially representative of a color of the optic nerve of a living eye.

8. The calibration device of claim 1, further comprising a lens positioned between the aperture and the mock retina, the lens magnifying or minifying the mock retina when the mock retina is viewed from outside the body.

9. The calibration device of claim 1, further comprising a mock iris configured to vary a size of the aperture.

10. The calibration device of claim 1, wherein the color of at least one of the regions has a red (R) value of between about 150 and 255 in an RGB color model.

11. The calibration device of claim 1, wherein the color of at least one of the regions has a red (R) value of between about 180 and 255 in an RGB color model.

12. The calibration device of claim 1, wherein the color of at least one of the regions has a red (R) value of between about 200 and 255 in an RGB color model.

13. The calibration device of claim 1, wherein the color of at least one of the regions has a red (R) value of greater than about 160, and green (G) and blue (B) values of less than about 100, in an RGB color model.

14. The calibration device of claim 1, wherein the color of at least one of the regions has a red (R) value of greater than about 200, and green (G) and blue (B) values of less than about 100, in an RGB color model.

15. An ophthalmic imaging system comprising:
the calibration device of claim 1; and
an eye fundus camera alignable with the calibration device.

16. The calibration device of claim 1, wherein the color model comprises a test target, wherein color frequencies of colors in the color model are determined from tones present in multiple human eyes.

17. A method, of calibrating an eye fundus camera, comprising:
positioning a body in front of a fundus camera, the body having an inner portion extending at least partially between a front and a back of the body;
imaging, with the camera and through an aperture at the front, an image model in the inner portion;
wherein the image model comprises a plurality of color regions, each of the regions having a color distinct from the color of another of the regions, wherein the color of each of the regions is substantially representative of a color of the fundus of a living eye; and
calibrating a color setting of the camera based on information derived from the imaging.

18. The method of claim 17, wherein the color of at least one of the regions is substantially representative of a color of the retina of a living eye.

19. The method of claim 17, wherein the color of at least one of the regions is substantially representative of a color of the optic nerve of a living eye.

20. The method of claim 17, wherein the image model comprises at least one of a dimensional or magnification scale, and further comprising calibrating a magnification setting of the camera based on information derived from the imaging.
